SEQUENCE LISTING (24 sequences)

SEQ ID NO: 1 - protein, 201 aa, Bos taurus
MARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 2 - protein, 224 aa, Bos taurus
MKVAFAAVLLICLVLSSSLFEVSMARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 3 - protein, 207 aa, Bos taurus
KNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 4 - protein, 232 aa, Bos taurus
MKVAFAAVLLICLVLSSSLFEVSMAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 5 - protein, 209 aa, Bos taurus
RELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 6 - protein, 234 aa, Bos taurus
MKVAFAAVLLICLVLSSSLFEVSMARELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 7 - protein, 209 aa, Bos taurus
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 8 - protein, 234 aa, Bos taurus
MKVAFAAVLLICLVLSSSLFEVSMARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 9 - protein, 169 aa, Bos taurus
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 10 - protein, 194 aa, Bos taurus
MKVAFAAVLLICLVLSSSLFEVSMAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 11 - protein, 564 aa, Bos taurus
LHLVLDLLPKLERSAAKPSGEPGCSCAQPAAEAAAPGWAQARGHPGGELEAAASAAGDAGWPNKHTLRILQDFSSDPSSNLTSHSLEKLPPAAEAAEGAPPGQDPGVRRPPDPAHRPLPRDPGPRGPVLPPGLSGDGSLLTRLFQHPLYQVPIPPLTEGDVLFNVNSDIRFNPKAATAENPDWPHEGPEDEFLPTGEAAVDSYPNWLKFHIGINRYELYSRHNPAVGALLQDLGTQKITSVAMKSGGTQLKLIMTFQNYGQALFKPMKQTREQETPPDFFYFSDYERHNAEIAAFHLDRILDFRRVPPVAGRLVNMTKEIRDVTRDKKLWRTFFISPANNVCFYGECSYYCSTEHALCGKPDQIEGSLAAFLPDLALAKRKTWRNPWRRSYHKRKKAEWEVDPDYCEEVRQTPPYDSSHRLLDVMDMTIFDFLMGNMDRHHYETFEKFGNETFIIHLDNGRGFGKHSHDELSILVPLQQCCRIRRSTYLRLQLLAQEEHRLSLLMAEALRADRVAPVLFQPHLEALDRRLRIVLRAVGDCVEKDGLHSVVEDDLGPEHRAAAGR

SEQ ID NO: 12 - protein, 589 aa, Bos taurus
MKVAFAAVLLICLVLSSSLFEVSMALHLVLDLLPKLERSAAKPSGEPGCSCAQPAAEAAAPGWAQARGHPGGELEAAASAAGDAGWPNKHTLRILQDFSSDPSSNLTSHSLEKLPPAAEAAEGAPPGQDPGVRRPPDPAHRPLPRDPGPRGPVLPPGLSGDGSLLTRLFQHPLYQVPIPPLTEGDVLFNVNSDIRFNPKAATAENPDWPHEGPEDEFLPTGEAAVDSYPNWLKFHIGINRYELYSRHNPAVGALLQDLGTQKITSVAMKSGGTQLKLIMTFQNYGQALFKPMKQTREQETPPDFFYFSDYERHNAEIAAFHLDRILDFRRVPPVAGRLVNMTKEIRDVTRDKKLWRTFFISPANNVCFYGECSYYCSTEHALCGKPDQIEGSLAAFLPDLALAKRKTWRNPWRRSYHKRKKAEWEVDPDYCEEVRQTPPYDSSHRLLDVMDMTIFDFLMGNMDRHHYETFEKFGNETFIIHLDNGRGFGKHSHDELSILVPLQQCCRIRRSTYLRLQLLAQEEHRLSLLMAEALRADRVAPVLFQPHLEALDRRLRIVLRAVGDCVEKDGLHSVVEDDLGPEHRAAAGR

SEQ ID NO: 13 - protein, 25 aa, Glycine max
MKVAFAAVLLICLVLSSSLFEVSMA

SEQ ID NO: 14 - DNA, 246 bp, Aspergillus nidulans
  source 1..246: mol_type = genomic DNA
cgggatagttccgacctaggattggatgcatgcggaaccgcacgagggcggggcggaaattgacacaccactcctctccacgcaccgttcaagaggtacgcgtatagagccgtatagagcagagacggagcactttctggtactgtccgcacgggatgtccgcacggagagccacaaacgagcggggccccgtacgtgctctcctaccccaggatcgcatccccgcatagctgaacatctatataa

SEQ ID NO: 15 - DNA, 33 bp, synthetic construct
  source 1..33: mol_type = other DNA
tacttagcccctccctcattgttaagggagcaa

SEQ ID NO: 16 - DNA, 444 bp, Glycine max
  source 1..444: mol_type = genomic DNA
agccctttttgtatgtgctaccccacttttgtctttttggcaatagtgctagcaaccaataaataataataataataatgaataagaaaacaaaggctttagcttgccttttgttcactgtaaaataataatgtaagtactctctataatgagtcacgaaacttttgcgggaataaaaggagaaattccaatgagttttctgtcaaatcttcttttgtctctctctctctctcttttttttttttctttcttctgagcttcttgcaaaacaaaaggcaaacaataacgattggtccaatgatagttagcttgatcgatgatatctttaggaagtgttggcaggacaggacatgatgtagaagactaaaattgaaagtattgcagacccaatagttgaagattaactttaagaatgaagacgtcttatcaggttcttcatgactt

SEQ ID NO: 17 - DNA, 504 bp, Glycine max
  source 1..504: mol_type = genomic DNA
tgagtaattctgatattagagggagcattaatgtgttgttgtgatgtggtttatatggggaaattaaataaatgatgtatgtacctcttgcctatgtaggtttgtgtgttttgttttgttgtctagctttggttattaagtagtagggacgttcgttcgtgtctcaaaaaaaggggtactaccactctgtagtgtatatggatgctggaaatcaatgtgttttgtatttgttcacctccattgttgaattcaatgtcaaatgtgttttgcgttggttatgtgtaaaattactatctttctcgtccgatgatcaaagttttaagcaacaaaaccaagggtgaaatttaaactgtgctttgttgaagattcttttatcatattgaaaatcaaattactagcagcagattttacctagcatgaaattttatcaacagtacagcactcactaaccaagttccaaactaagatgcgccattaacatcagccaataggcattttcagcaa

SEQ ID NO: 18 - DNA, 766 bp, Agrobacterium tumefaciens
  source 1..766: mol_type = genomic DNA
gtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacct

SEQ ID NO: 19 - DNA, 642 bp, Pissum sativum
  source 1..642: mol_type = genomic DNA
agctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaa

SEQ ID NO: 20 - protein, 299 aa, Homo sapiens
AIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 21 - protein, 24 aa, Glycine max
MKVAFAAVLLICLVLSSSLFEVSM

SEQ ID NO: 22 - protein, 323 aa, Homo sapiens
MKVAFAAVLLICLVLSSSLFEVSMAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 23 - DNA, 972 bp, Homo sapiens
  source 1..972: mol_type = genomic DNA
atgaaggttgcattcgccgctgttctactcatatgtcttgtgttgtcatcttctttgttcgaggtgagtatggctatcccagttaaacaagctgatagtggaagcagtgaagagaaacagctctacaacaagtacccggatgcagttgctacatggttgaaccctgacccctcacagaagcaaaatctgttagcgccacaaaacgctgtctcttccgaagaaacaaacgactttaagcaggaaactctcccgtcaaagtctaatgagtctcatgatcacatggacgatatggacgacgaggatgatgacgatcacgtcgattcccaagacagcatagattcaaatgattcagacgacgtggatgacacagacgactcacatcaatcagacgaaagccatcatagtgacgaaagcgacgaacttgtgacagacttccctacagacttaccggctactgaggtgttcactcccgtcgtgcctaccgtggacacctatgatggtagaggagactccgtcgtgtatgggctgcgtagtaagtccaagaagtttcgtagaccagacatacagtatcctgacgctacagacgaagatataacttctcacatggagtccgaagagttgaatggggcgtataaagcaatacctgtggctcaggatttaaatgccccaagcgattgggactcccgcggtaaagattcatatgaaacgtcacaattagatgaccagagtgcggaaacacactcccacaagcagtcacgtttgtataaacgcaaagctaatgacgagagcaatgagcactcagacgtcatagactctcaagagttatcaaaggtttctcgtgaattccactctcatgagttccactcccatgaggatatgcttgtggttgaccccaaaagtaaagaggaagacaaacatctaaaattcaggataagtcatgaacttgacagcgcctcatctgaggtgaactaa

SEQ ID NO: 24 - protein, 821 aa, Aspergillus nidulans
MADTRRRQNHSCDPCRKGKRRCDAPENRNEANENGWVSCSNCKRWNKDCTFNWLSSQRSKAKGAAPRARTKKARTATTTSEPSTSAATIPTPESDNHDAPPVINSHDALPSWTQGLLSHPGDLFDFSHSAIPANAEDAANVQSDAPFPWDLAIPGDFSMGQQLEKPLSPLSFQAVLLPPHSPNTDDLIRELEEQTTDPDSVTDTNSVQQVAQDGSLWSDRQSPLLPENSLCMASDSTARRYARSTMTKNLMRIYHDSMENALSCWLTEHNCPYSDQISYLPPKQRAEWGPNWSNRMCIRVCRLDRVSTSLRGRALSAEEDKAAARALHLAIVAFASQWTQHAQRGAGLNVPADIAADERSIRRNAWNEARHALQHTTGIPSFRVIFANIIFSLTQSVLDDDEQHGMGARLDKLLENDGAPVFLETANRQLYTFRHKFARMQRRGKAFNRLPGGSVASTFAGIFETPTPSSESPQLDPVVASEEHRSTLSLMFWLGIMFDTLSAAMYQRPLVVSDEDSQISSASPPRRGAETPINLDCWEPPRQVPSNQEKSDVWGDLFLRTSDSLPDHESHTQISQPAARWPCTYEQAAAALSSATPVKVLLYRRVTQLQTLLYRGASPARLEAAIQRTLYVYNHWTAKYQPFMQDCVANHELLPSRIQSWYVILDGHWHLAAMLLADVLESIDRDSYSDINHIDLVTKLRLDNALAVSALARSSLRGQELDPGKASPMYRHFHDSLTEVAFLVEPWTVVLIHSFAKAAYILLDCLDLDGQGNALAGYLQLRQNCNYCIRALQFLGRKSDMAALVAKDLERGLNGKVDSFL

(19) United States
(12) Patent Application Publication
(10) Pub. No.: US 2025/0042965 A1
(43) Pub. Date: Feb. 6, 2025

(54) HUMAN OSTEOPONTIN PROTEIN PRODUCTION IN PLANT SYSTEMS

(71) Applicant: THE REGENTS OF THE UNIVERSITY OF COLORADO A BODY CORPORATE, Denver, CO (US)

(72) Inventors: Brian DEDECKER, Boulder, CO (US); Simon KALMUS, Boulder, CO (US)

(21) Appl. No.: 18/925,427

(22) Filed: Oct. 24, 2024

Related U.S. Application Data
(63) Continuation-in-part of application No. PCT/US2023/066313, filed on Apr. 27, 2023.
(60) Provisional application No. 63/335,288, filed on Apr. 27, 2022.

(51) Int. Cl. C07K 14/52; C12N 15/82

(52) U.S. Cl. CPC C07K 14/52; C12N 15/8242

(57) ABSTRACT

The present invention includes novel systems, methods, and compositions for the non-endogenous production of human Osteopontin in plants, and preferably Glycine max or other high-biomass crops.

### CROSS-REFERENCE TO RELATED APPLICATIONS

This application is a continuation in part of PCT Application No. PCT/US23/66313 having an international filing date of Apr. 27, 2023, which PCT application claimed the benefit of U.S. Application Ser. No. 63/335,288, filed Apr. 27, 2022, both of which are incorporated by reference in their entirety.

### SEQUENCE LISTING

The instant application contains contents of the electronic sequence listing (90245.00772-Sequence-Listing.xml; Size: 29,921 bytes; and Date of Creation: Oct. 24, 2024) is herein incorporated by reference in its entirety.

### FIELD OF INVENTION

The present invention relates to the non-endogenous production of proteins in plant systems. In particular, the present invention relates to novel systems, methods and compositions for the production of Human Osteopontin (hOPN) in transgenic plants, namely Glycine Max (soybeans).

### BACKGROUND

Human Osteopontin (hOPN) is a multifunctional bioactive protein that is implicated in numerous biological processes, such as bone remodeling, inhibition of ectopic calcification, and cellular adhesion and migration, as well as several immune functions. hOPN has cytokine-like properties and is a key factor in the initiation of T helper 1 immune responses. Osteopontin comprises several structural domains, some of which include an integrin-binding (RGD) adhesive domain (Arg-Gly-Asp sequence), and aspartic acid rich calcium binding regions. hOPN is subject to numerous post translational modifications, including thrombin cleavage, sulfation, glycosylation, trans-glutamination, and phosphorylation. hOPN is present in most tissues and body fluids, with the highest concentrations being found in milk. Recent studies have shown that supplementation of hOPN in infant formula can affect immune functions, and intestinal development in the newborn as well as brain development in infants. For example, hOPN added to infant formula shifted overall gene expression in the intestinal transcriptome differences toward a profile more similar to that in breastfed infants on the intestinal transcriptome microarray analyses showed a large number of genes that were differentially expressed between formula-fed and breastfed infants. Despite these benefits, methods to produce hOPN at commercial scale have proven ineffective, leaving a long-felt need for a practical production-system for the same.

Producing hOPN from cows is expensive, environmentally unsustainable and inhumane. Producing hOPN in soybeans will alleviate all three of these concerns.

### SUMMARY OF THE INVENTION

In one aspect, the present invention relates to the nonendogenous, (also referred to generally as heterologous) production of commercially relevant compounds in transgenic plants. In particular, the present invention relates to novel systems, methods and compositions for the production of hOPN in transgenic plants, namely Glycine Max (soybeans). In alternative embodiments, the systems and methods of the invention could also be used in plants, and preferably seeds harvested from commonly farmed crops such as corn, rice and rapeseeds.

In another preferred aspect, present invention further includes novel, systems, methods, and compositions for engineering plants, and preferably soybean plants to produce hOPN. In a preferred embodiment, the present invention may include novel, systems, methods, and compositions for engineering a plant, such as a soybean plant to heterologously express a nucleotide sequence encoding a human Osteopontin, also sometimes referred to as hOPN.

In another preferred aspect, the invention includes novel, systems, methods, and compositions for engineering a plant, such as a soybean plants to heterologously express a nucleotide sequence encoding a chimeric peptide having a first domain encoding a modified hOPN peptide where its native localization signal has been disrupted or removed and replaced with a second domain encoding a recombinant localization signal, that can include a heterologous localization signal, or a localization signal that is endogenous to the plant cell in which it will be expressed.

In a preferred aspect, this heterologous localization signal can facilitate the export of the modified hOPN out of the plant cell's protoplast and be localized to the apoplast of a plant cell. Additional aspects include isolation of the hOPN which can include washing the heterologous peptide into an aqueous supernatant and further separated from the plant cells by centrifugation.

In another preferred aspect, expression of a nucleotide sequence encoding a modified hOPN can be subject to an inducible promoter system, such that expression is not induced in the plant biomass, but instead is induced in the seed of a plant, for example during germination.

Additional aspects of the inventive technology will be evident from the detailed description and figures presented below.

### BRIEF DESCRIPTION OF THE FIGURES

FIG. 1: SDS-PAGE Western Blot analysis of 10 mg soybean tissue. Primary Abcam Anti-Human Osteopontin (SPP1) polyclonal rabbit antibody with secondary anti-rabbit polyclonal goat HRP antibody. Lane 1—Control wild type soybeans lacking expression of human Osteopontin. Lane 2—Engineered soybean tissue expressing recombinant human Osteopontin modified with a signal peptide sequence from soybean Extensin. Predicted band sizes: 35 kDa without glycosylation and phosphorylation or 60 kDa with both modifications. Observed band size: 60 kDa, indicating proper cellular processing of engineered human Osteopontin with post-translational modifications (glycosylation and phosphorylation) in soybeans.

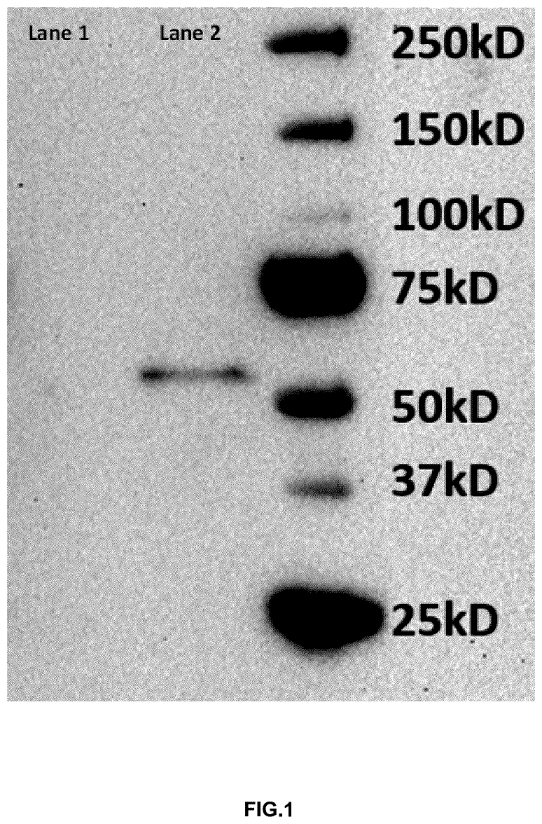

### DETAILED DESCRIPTION OF THE INVENTION

Disclosed herein are novel systems, methods, and compositions for the heterologous production of human Osteopontin in a plant cell. In this embodiment, transgenic plant or seed of the invention may include a plant or seed, and preferably a soybean plant or seed, expressing a heterologous nucleotide sequence encoding a hOPN gene, or a fragment of variant thereof. In this embodiment, the invention includes a transformed plant cell, such as a transformed plant seed cell, expressing a heterologous nucleotide sequence, operably linked to a promoter, encoding a hOPN peptide according to SEQ ID NO. 20, or a fragment of variant thereof.

As used herein, “osteopontin” refers to a secreted phosphoprotein having an apparent molecular weight of 44 kDa that is highly negatively charged and frequently associated with mineralization processes, and that contains the amino acid sequence Arg-Gly-Asp (RGD). As used herein, osteopontin includes, but need not be limited to, the proteins described in Oldberg et al., 1986, Proc. Natl. Acad. Sci. U.S.A. 83:8819-23; Craig et al., 1989, J. Biol. Chem. 264:9682-89; Cantor et al., U.S. Pat. No. 5,049,659, issued Sep. 17, 1991, each of which is specifically incorporated herein by reference, as well as to homologous proteins from other species.

In another preferred embodiment, the heterologous nucleotide sequence encoding a hOPN peptide of the invention can include a heterologous nucleotide sequence that is codon optimized for expression in a plant cell, and preferably a soybean plant cell. As noted above, the heterologous nucleotide sequence encoding the non-endogenous hOPN may be subject to an inducible promoter, such as an AlcA/AlcR inducible expression system. The non-endogenous hOPN may be encoded and expressed in a plant cell, and preferably a soybean plant cell, by a heterologous nucleotide sequence comprising one or more expression cassettes, operably linked to promoter(s), encoding a hOPN peptide according to SEQ ID NO. 20, or a fragment of variant thereof.

In additional embodiments, invention further includes a transformed plant cell, such as a transformed plant seed cell, expressing a heterologous nucleotide sequence, operably linked to a promoter, encoding a chimeric hOPN peptide having an endogenous signal peptide configured allow the export of the peptide out of the cell. Specifically, the signal peptide of the invention facilitates the export of the chimeric hOPN peptide outside of the plant cell where it can be localized in the apoplast which is positioned between the plasma membrane and the cell wall of the transformed plant cell.

In additional embodiments, invention further includes a transformed plant cell, such as a transformed plant seed cell, expressing a heterologous nucleotide sequence, operably linked to a promoter, encoding a chimeric peptide having a first and second domain. The first domain of the chimeric peptide includes a hOPN peptide domain according to SEQ ID NO. 20, or a fragment of variant thereof where the proteins native localization signal has been disrupted or removed and replaced with a second domain encoding, preferably a localization signals endogenous to the plant to be transformed. Here, the second domain of the chimeric peptide includes an N-linked extensin signal peptide derived from Glycine Max according to SEQ ID NO. 21, or a fragment of variant thereof linked to the first domain. In this embodiment, the plant cell of the invention can be transformed to expressing a heterologous nucleotide sequence, operably linked to preferably an inducible promoter, encoding a chimeric peptide having a first and second domain forming an exportable hOPN peptide, sometime referred to as modified hOPN or hOPN-M, according to SEQ ID NO. 22, or a fragment of variant thereof. In certain embodiments, the first and second domains can be joined by a linker, such as a small peptide sequence or other common domain linker sequences known in the art. In this configuration, the heterologously expressed hOPN-M can be localized to the apoplast of a plant cell where it can be washed into an aqueous supernatant and further separated from the plant cells by centrifugation, among other separation methods known by those or ordinary skill in the art. The isolated can further be processed to remove the localization signal, and/or can be used as a constituent component for one or more commercial products, such a supplement for infant formula.

In a preferred embodiment, the heterologous nucleotide sequence encoding a hOPN-M chimeric peptide of the invention can include a heterologous nucleotide sequence that is codon optimized for expression in a plant cell, and preferably a soybean plant cell according to SEQ ID NO. 23, or a sequence having at least 80% sequence homology with SEQ ID NO. 23. As noted above, the heterologous nucleotide sequence encoding the non-endogenous hOPN may be subject to an inducible promoter, such as an AlcA/AlcR inducible expression system. The non-endogenous hOPN-M may be encoded and expressed in a plant cell, and preferably a soybean plant cell, by a heterologous nucleotide sequence comprising one or more expression cassettes, operably linked to promoter(s), encoding a hOPN-M peptide according to SEQ ID NO. 22, or a fragment of variant thereof. In a preferred embodiment, the heterologously expressed coding sequences of the invention may be driven by an inducible promoter, such as the inducible AlcA/AlcR system with the AlcA promoter sequence according to SEQ ID NO. 14, and a AlcR protein according to SEQ ID NO. 24, which responds to ethanol and binds to the fungal AlcA promoter in its presence. A 33 base pair spacer (SEQ ID NO. 15) may be utilized between the transcription start site and translation start site for expression of hONP-M. Terminators according to SEQ ID NO. 16-19 may be used to comprise the 3′ untranslated regions (3′ UTR) of the transgenes as described herein.

A polypeptide can be expressed in monocot plants and/or dicot plants. Techniques for introducing nucleic acids into plants are known in the art, and include, without limitation, Agrobacterium-mediated transformation, viral vector-mediated transformation, electroporation, and particle gun transformation (also referred to as biolistic transformation). See, for example, U.S. Pat. Nos. 5,538,880; 5,204,253; 6,329,571; and U.S. Pat. No. 6,013,863; Richards et al., Plant Cell. Rep. 20:48-20 54 (2001); Somleva et al., Crop Sci. 42:2080-2087 (2002); Sinagawa-Garcia et al., Plant Mol Biol (2009) 70:487-498; and Lutz et al., Plant Physiol., 2007, Vol. 145, pp. 1201-1210. In some instances, intergenic transformation of plastids can be used as a method of introducing a polynucleotide into a plant cell. In some instances, the method of introduction of a polynucleotide into a plant comprises chloroplast transformation. In some instances, the leaves and/or stems can be the target tissue of the introduced polynucleotide. If a cell or cultured tissue is used as the recipient tissue for transformation, plants can be regenerated from transformed cultures if desired, by techniques known to those skilled in the art.

Other suitable methods for introduce polynucleotides include electroporation of protoplasts, polyethylene glycol-mediated delivery of naked DNA into plant protoplasts, direct gene transformation through imbibition (e.g., introducing a polynucleotide to a dehydrated plant), transformation into protoplasts (which can comprise transferring a polynucleotide through osmotic or electric shocks), chemical transformation (which can comprise the use of a polybrene-spermidine composition), microinjection, pollen-tube pathway transformation (which can comprise delivery of a polynucleotide to the plant ovule), transformation via liposomes, shoot apex method of transformation (which can comprise introduction of a polynucleotide into the shoot and regeneration of the shoot), sonication-assisted agrobacterium transformation (SAAT) method of transformation, infiltration (which can comprise a floral dip, or injection by syringe into a particular part of the plant (e.g., leaf)), silicon-carbide mediated transformation (SCMT) (which can comprise the addition of silicon carbide fibers to plant tissue and the polynucleotide of interest), electroporation, and electrophoresis. Such expression may be from transient or stable transformations.

The term “homolog” or “variant,” used with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species. Most often, homologs or variant will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes. A “fragment” used with respect to an original enzyme or gene refers to a truncated portion of the peptide or gene that still retains its intended function.

As used herein, the term “sequence identity” with regard to a contiguous nucleic acid sequence, refers to contiguous nucleotide sequences that hybridize under appropriate conditions to the reference nucleic acid sequence. For example, homologous sequences may have from about 70%-100, or more generally 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired, for example, under stringent hybridization conditions.

The term “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. An “inducible” promoter may be a promoter which may be under environmental control. Tissue-specific, tissue-preferred, cell type specific, and inducible promoters constitute the class of “non-constitutive” promoters. A “constitutive” promoter is a promoter which may be active under most environmental conditions or in most cell or tissue types.

As used herein, the term “transformation” or “genetically modified” refers to the transfer of one or more nucleic acid molecule(s) into a cell. A plant is “transformed” or “genetically modified” by a nucleic acid molecule transduced into the plant when the nucleic acid molecule becomes stably replicated by the plant. As used herein, the term “transformation” or “genetically modified” encompasses all techniques by which a nucleic acid molecule can be introduced into, such as a plant.

The terms “transgenic,” “transformed,” “transformation,” and “transfection” are similar in meaning to “recombinant.” “Transformation,” “transgenic,” and “transfection” refer to the transfer of a polynucleotide into the genome of a host organism or into a cell. Such a transfer of polynucleotides can result in genetically stable inheritance of the polynucleotides or in the polynucleotides remaining extra-chromosomally (not integrated into the chromosome of the cell). Genetically stable inheritance may potentially require the transgenic organism or cell to be subjected for a period of time to one or more conditions which require the transcription of some or all of the transferred polynucleotide in order for the transgenic organism or cell to live and/or grow. Polynucleotides that are transformed into a cell but are not integrated into the host's chromosome remain as an expression vector within the cell. One may need to grow the cell under certain growth or environmental conditions in order for the expression vector to remain in the cell or the cell's progeny. Further, for expression to occur the organism or cell may need to be kept under certain conditions. Host organisms or cells containing the recombinant polynucleotide can be referred to as “transgenic” or “transformed” organisms or cells or simply as “transformants,” as well as recombinant organisms or cells.

A genetically altered organism is any organism with any change to its genetic material, whether in the nucleus or cytoplasm (organelle). As such, a genetically altered organism can be a recombinant or transformed organism. A genetically altered organism can also be an organism that was subjected to one or more mutagens or the progeny of an organism that was subjected to one or more mutagens and has changes in its DNA caused by the one or more mutagens, as compared to the wild-type organism (i.e., organism not subjected to the mutagens). Also, an organism that has been bred to incorporate a mutation into its genetic material is a genetically altered organism. For the purposes of this invention, the organism is a plant.

As used herein, the term “endogenous” refers to any material from or produced inside an organism, cell, tissue or system.

As used herein, the term “exogenous” or “heterologous” refers to any material introduced from or produced outside an organism, cell, tissue or system.

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; or can be regulatory in nature, etc. There are various types of vectors including virus, plasmid, bacteriophages, cosmids, and bacteria. An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of a cassette assists in the assembly of the expression vectors. An expression vector is a replicon, such as plasmid, phage, virus, chimeric virus, or cosmid, and which contains the desired polynucleotide sequence operably linked to the expression control sequence(s).

As is known in the art, different organisms preferentially utilize different codons for generating polypeptides. Such “codon usage” preferences may be used in the design of nucleic acid molecules encoding the proteins and chimeras of the invention in order to optimize expression in a particular host cell system. For example, all nucleotides of the present invention may be optimized for expression in a select organisms such a Glycine Max.

A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. The Table below, contains information about which nucleic acid codons encode which amino acids.

### Amino Acid Nucleic Acid Codons

| Amino Acid | Nucleic Acid Codons |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

Moreover, because the proteins are described herein, one can chemically synthesize a polynucleotide which encodes these polypeptides/chimeric proteins. Oligonucleotides and polynucleotides that are not commercially available can be chemically synthesized e.g., according to the solid phase phosphoramidite triester method first described by Beaucage and Caruthers, Tetrahedron Letts. 22:1859-1862 (1981), or using an automated synthesizer, as described in Van Devanter et al., Nucleic Acids Res. 12:6159-6168 (1984). Other methods for synthesizing oligonucleotides and polynucleotides are known in the art. Purification of oligonucleotides is by either native acrylamide gel electrophoresis or by anion-exchange HPLC as described in Pearson & Reanier, J. Chrom. 255:137-149 (1983).

The term “plant” or “plant system” includes whole plants, plant organs, progeny of whole plants or plant organs, embryos, somatic embryos, embryo-like structures, protocorms, protocorm-like bodies (PLBs), and suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The class of plants that can be used in the method of the invention is generally as broad as the class of higher and lower plants amenable to the molecular biology and plant breeding techniques described herein, specifically angiosperms (monocotyledonous (monocots) and dicotyledonous (dicots) plants including cudicots. It includes plants of a variety of ploidy levels, including aneuploid, polyploid, diploid, haploid and hemizygous. In one preferred embodiment, the genetically altered plants described herein can be dicot crops, such as soybean.

The term “expression,” as used herein, or “expression of a coding sequence” (for example, a gene or a transgene) refer to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex. The term “ribonucleic acid” (RNA) is inclusive of iRNA (inhibitory RNA), dsRNA (double stranded RNA), siRNA (small interfering RNA), mRNA (messenger RNA), miRNA (micro-RNA), hpRNA (hairpin RNA), IRNA (transfer RNA), whether charged or discharged with a corresponding acetylated amino acid), and cRNA (complementary RNA). The term “deoxyribonucleic acid” (DNA) is inclusive of cDNA, genomic DNA, and DNA-RNA hybrids. The terms “nucleotide sequence” and “nucleotide sequence segment,” or more generally “sequence,” will be understood by those in the art as a functional term that includes both genomic sequences, ribosomal RNA sequences, transfer RNA sequences, messenger RNA sequences, operon sequences, and smaller engineered nucleotide sequences that encoded or may be adapted to encode, peptides, polypeptides, or proteins.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide. It should be noted that any reference to a SEQ ID, or sequence specifically encompasses that sequence, as well as all corresponding sequences that correspond to that first sequence. For example, for any amino acid sequence identified, the specific specifically includes all compatible nucleotide (DNA and RNA) sequences that give rise to that amino acid sequence or protein, and vice versa.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hair-pinned, circular, and padlocked conformations.

The term “sequence identity” or “identity,” as used herein in the context of two nucleic acid or polypeptide sequences, refers to the residues in the two sequences that are the same when aligned for maximum correspondence over a specified comparison window.

The terms “approximately” and “about” refer to a quantity, level, value, or amount that varies by as much as 30%, or in another embodiment by as much as 20%, and in a third embodiment by as much as 10% to a reference quantity, level, value or amount. As used herein, the singular form “a,” “an,” and “the” include plural references unless the context clearly dictates otherwise. As used herein the term “increased, or decreased with respect to the use or effect of an antimicrobial peptide means increased, or decreased compared to wild-type.

As used herein, the singular form “a,” “an,” and “the” include plural references unless the context clearly dictates otherwise. For example, the term “a peptide” includes both a single peptide and a plurality of peptides.

As defined herein, with respect to any antimicrobial peptide the terms “derived from” or “from” means directly isolated or obtained from a particular source or alternatively having identifying characteristics of a substance or organism isolated or obtained from a particular source. In the event that the “source” is an organism, “derived from” or “from” means that it may be isolated or obtained from the organism itself or from the medium used to culture or grow said organism.

As used herein, “heterologous” or “exogenous” in reference to a nucleic acid is a nucleic acid that originates from a foreign species, or is synthetically designed, or, if from the same species, is substantially modified from its native form in composition and/or genomic locus by deliberate human intervention. A heterologous protein may originate from a foreign species or, if from the same species, is substantially modified from its original form by deliberate human intervention.

As used herein, a “host cell” means a cell which contains an introduced nucleic acid construct and supports the replication and/or expression of the construct.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention. The term “stereoisomer” refers to a molecule that is an enantiomer, diastereomer or geometric isomer of a molecule. Stereoisomers, unlike structural isomers, do not differ with respect to the number and types of atoms in the molecule's structure but with respect to the spatial arrangement of the molecule's atoms. Examples of stereoisomers include the (+) and (−) forms of optically active molecules.

### REFERENCES

1. Alshamiry, F., & Abdelrahman, M. (2020). Review: Milk protein, types and structure, synthesis of casein.
2. Hohmann, L. G., Yin, T., Schweizer, H., Giambra, I. J., König, S., & Scholz, A. M. (2020). Comparative Effects of Milk Containing A1 versus A2 β-Casein on Health, Growth and β-Casomorphin-7 Level in Plasma of Neonatal Dairy Calves. Animals: An Open Access Journal from MDPI, 11 (1), E55.
3. Ishikawa, H. O., Xu, A., Ogura, E., Manning, G., & Irvine, K. D. (2012). The Raine Syndrome Protein FAM20C Is a Golgi Kinase That Phosphorylates Bio-Mineralization Proteins. PLOS ONE, 7 (8), e42988.
4. Philip, R., Darnowski, D. W., Maughan, P. J., & Vodkin, L. O. (2001). Processing and localization of bovine beta-casein expressed in transgenic soybean seeds under control of a soybean lectin expression cassette. Plant Science: An International Journal of Experimental Plant Biology, 161 (2), 323-335.
5. Sadiq, U., Gill, H., & Chandrapala, J. (2021). Casein Micelles as an Emerging Delivery System for Bioactive Food Components. Foods, 10 (8), 1965.

What is claimed is:

1. A composition comprising a chimeric peptide having a first domain and a second domain, wherein said first domain encodes an osteopontin (OPN) peptide, or a fragment or variant thereof, having its localization signal disrupted or removed, and said second domain encodes a recombinant localization signal configured to export the chimeric peptide out of a plant cell.

2. The composition of claim 1, wherein said recombinant localization signal comprises an extensin signal peptide.

3. The composition of claim 2, wherein said extensin signal peptide comprises a peptide according to SEQ ID NO. 21, or a fragment or variant thereof.

4. The composition of claim 1, wherein said OPN peptide comprises a peptide according to SEQ ID NO. 20, wherein the localization signal has been disrupted or removed, or a fragment or variant thereof.

5. The composition of claim 1, wherein said chimeric peptide comprises a peptide according to SEQ ID NO. 22, or a fragment or variant thereof.

6. A plant cell configured to express a heterologous nucleotide sequence, operably linked to a promoter, encoding a chimeric peptide having a first domain and a second domain, wherein said first domain encodes an osteopontin (OPN) peptide, or a fragment or variant thereof, having its localization signal disrupted or removed, and said second domain encodes a recombinant localization signal configured to export the chimeric peptide out of said plant cell.

7. The plant cell of claim 6, wherein said plant cell comprises a soybean plant or seed.

8. The plant cell of claim 6, wherein said promoter comprises an inducible promoter.

9. The plant cell of claim 8, wherein said inducible promoter comprises an ALCR/alcA ethanol switch.

10. The plant cell of claim 6, wherein the heterologous nucleotide sequence comprises nucleotide sequence according to SEQ ID NO. 23, or a sequence having at least 80% homology with SEQ ID NO. 23.

11. The plant cell of claim 6, wherein said recombinant localization signal comprises an extensin signal peptide.

12. The plant cell of claim 11, wherein said extensin signal peptide comprises a peptide according to SEQ ID NO. 21, or a fragment or variant thereof.

13. The plant cell of claim 6, wherein said OPN peptide comprises a peptide according to SEQ ID NO. 20, wherein the localization signal has been disrupted or removed, or a fragment or variant thereof.

14. The plant cell of claim 6, wherein said chimeric peptide comprises a peptide according to SEQ ID NO. 22, or a fragment or variant thereof.

15. The plant cell of claim 6, wherein said nucleotide sequence is codon optimized for expression in Glycine Max.

16. The plant cell of claim 6, wherein said nucleotide sequence comprises a nucleotide sequence according to SEQ ID NO. 23, or a sequence having at least 80% homology with SEQ ID NO. 23.

17. The plant cell of claim 6, wherein said nucleotide sequence comprises a nucleotide sequence encoding the chimeric peptide according to SEQ ID NO. 22, or fragment or variant thereof.
